(19) United States
(12) Patent Application Publication
(10) Pub. No.: US 2025/0345118 A1
(43) Pub. Date: Nov. 13, 2025

(54) DESIGN METHOD OF ANATOMICAL PLATE FOR TREATING TIBIAL PLATEAU FRACTURE AND INTERNAL FIXATION DEVICE

(71) Applicants: THE FOURTH MEDICAL CENTER OF PLA GENERAL HOSPITAL, Beijing (CN); Beijing Libeier Bio-engineering Institute Co., Ltd., Beijing (CN)

(72) Inventors: Wei Zhang, Beijing (CN); Bowen Yuan, Beijing (CN); Meng Li, Beijing (CN); Jiantao Li, Beijing (CN); Jian Li, Beijing (CN); Zicheng Zhang, Beijing (CN); Mengmeng Hu, Beijing (CN); Dong Zhang, Beijing (CN); Ting Li, Beijing (CN); Mingli Liu, Beijing (CN); Xiaomeng Ren, Beijing (CN); Yi Gao, Beijing (CN); Longbo Du, Beijing (CN); Rui Ma, Beijing (CN); Yuchen Guo, Beijing (CN)

(21) Appl. No.: 19/072,705

(22) Filed: Mar. 6, 2025

(30) Foreign Application Priority Data
May 7, 2024 (CN) 202410553763.2

(51) Int. Cl. A61B 34/10; A61B 17/80

(52) U.S. Cl. CPC A61B 34/10; A61B 17/8061; A61B 2034/102; A61B 2034/105; A61B 2034/108

(57) ABSTRACT

A design method of an anatomical plate for treating a tibial plateau fracture and an internal fixation device are provided. The design method includes: obtaining a high-precision three-dimensional model of a fractured bone of a patient; obtaining a post-reduction bone model; extracting point arrays of a cross section and a sagittal plane and obtaining solid body designs of a lateral plate body and a medial plate body; and adjusting thicknesses of proximal ends of the lateral plate body and the medial plate body, adjusting thicknesses of main bodies of the lateral plate body and the medial plate body, cutting the lateral plate body and the medial plate body from a sagittal plane according to a fit condition of the lateral plate body and the medial plate body, and determining a position and a direction of a screw hole to obtain a lateral anatomical plate and a medial anatomical plate.

### CROSS-REFERENCE TO RELATED APPLICATION

This application claims priority of Chinese Patent Application No. 202410553763.2 filed on May 7, 2024, the entire contents of which are incorporated herein by reference.

### TECHNICAL FIELD

The present disclosure relates to the technical field of medical apparatus and instruments, and in particular, to a design method of an anatomical plate for treating a tibial plateau fracture and an internal fixation device.

### BACKGROUND

Fractures are common in osteopathic medicine, where tibial plateau fractures have drawn great attention for high complexity. The tibial plateau fractures are usually caused by high-energy traumas, such as car accidents and tumbles, and thus are important treatment challenges in orthopedic emergencies. The clinical manifestations of the tibial plateau fracture include obvious fracture dislocation, soft tissue injury, and joint instability.

According to medical documents, the probability of occurrence of the tibial plateau fracture accounts for 5% to 10% among all fractures, where Schatzker type V and type VI fractures take up a great proportion in this ratio. The type V fracture commonly occurs in high-speed car accidents. Bone fragments are created by violent impact, leading to dislocation of a plurality of parts of the tibial plateau. The type VI fracture is rare, and is severe because it not only involves the tibial plateau fracture but also is accompanied by joint dislocation. Therefore, the type VI fracture usually requires emergency intervention.

In the related art, it is difficult for a traditional internal fixation system (e.g., a traditional fixation way with a plate and a screw) to meet the stable internal fixation requirement. For example, type V and type VI fractures often need to be fixed with 3 to 4 plates; the soft tissue tension is high and irritation is serious. In more severe cases, the skin could not be closed, leading to necrosis and even amputation. Furthermore, for posterolateral plateau fracture, there is currently no effective internal fixation system, and plates at other parts are often needed to assist with internal fixation. The fit is not good enough, and it is easy to cause poor maintenance of fracture alignment, affecting the internal fixation effect.

### SUMMARY

The present disclosure provides a design method of an anatomical plate for treating a tibial plateau fracture and an internal fixation device to solve the problems of lack of an effective internal fixation system, a large number of plates applied, unstable internal fixation effect, and the like that may affect the healing process of a fracture in the related art.

According to one aspect of the present disclosure, there is a design method of an anatomical plate for treating a tibial plateau fracture. The design method of an anatomical plate for treating a tibial plateau fracture includes: obtaining a high-precision three-dimensional model of a fractured bone of a patient; simulating intraoperative reduction on the high-precision three-dimensional model to obtain a post-reduction bone model; extracting point arrays of a cross section and a sagittal plane from medial and lateral edges and medial and lateral diaphysis parts of a tibial plateau of the post-reduction bone model, connecting the point arrays of the cross section and the sagittal plane to obtain a connection curve, and obtaining solid body designs of a lateral plate body and a medial plate body according to the connection curve; and adjusting thicknesses of proximal ends of the lateral plate body and the medial plate body according to a condition of soft tissue of the patient, adjusting thicknesses of main bodies of the lateral plate body and the medial plate body according to a body weight of the patient, cutting the lateral plate body and the medial plate body from a sagittal plane according to a fit condition of the lateral plate body and the medial plate body, and determining a position and a direction of a screw hole to obtain a lateral anatomical plate and a medial anatomical plate.

Further, the cutting the lateral plate body and the medial plate body from a sagittal plane according to a fit condition of the lateral plate body and the medial plate body may include: cutting the lateral plate body into a T shape in the sagittal plane, and designing both wings of the lateral plate body forming a C-shaped cross section; and cutting the medial plate body into a γ shape in the sagittal plane, and designing both wings of the medial plate body to extend inwards and a middle part of the medial plate body to be a recess for escaping medial condyle process.

Further, the cutting the lateral plate body and the medial plate body from a sagittal plane according to a fit condition of the lateral plate body and the medial plate body may further include: designing an upper edge of the lateral plate body to be flush with an upper edge of the tibial plateau; and designing an upper edge of the medial plate body to be lower than the upper edge of the lateral plate body.

Further, the determining a position and a direction of a screw hole may include: designing two rows of first screw holes at the proximal end of the lateral plate body, designing an obliquely upward second screw hole at a first screw hole in a neck of the lateral plate body, and designing a third screw hole in the main body of the lateral plate body.

Further, the determining a position and a direction of a screw hole may further include: designing two rows of fourth screw holes extending obliquely backwards from front at a front side of the medial plate body, designing a fifth screw hole at a rear side of the medial plate body, designing an obliquely upward sixth screw hole at a first screw hole in a neck of the medial plate body, and designing a seventh screw hole in the main body of the medial plate body.

Further, the determining a position and a direction of a screw hole may further include: designing an included angle between an extension direction of the fourth screw hole and a horizontal axis of the tibial plateau to be less than or equal to 30°; and/or designing the two rows of first screw holes and the two rows of fourth screw holes to be staggered up and down.

Further, the adjusting thicknesses of proximal ends of the lateral plate body and the medial plate body according to a condition of soft tissue of the patient may include: if a thickness of the soft tissue of the patient is less than 20 mm, setting the thicknesses of the proximal ends of the lateral plate body and the medial plate body to be 2 mm to 2.5 mm; if the thickness of the soft tissue of the patient is 20 mm to 30 mm, setting the thicknesses of the proximal ends of the lateral plate body and the medial plate body to be 2.5 mm to 3 mm; and if the thickness of the soft tissue of the patient is greater than 30 mm, setting the thicknesses of the proximal ends of the lateral plate body and the medial plate body to be 3 mm to 3.5 mm.

Further, the adjusting thicknesses of main bodies of the lateral plate body and the medial plate body according to a body weight of the patient may include: if the body weight of the patient is less than 60 kg, setting the thicknesses of the main bodies of the lateral plate body and the medial plate body to be 3 mm to 3.5 mm; if the body weight of the patient is greater than or equal to 60 kg and less than or equal to 90 kg, setting the thicknesses of the main bodies of the lateral plate body and the medial plate body to be 3.5 mm to 4 mm; and if the body weight of the patient is greater than 90 kg, setting the thicknesses of the main bodies of the lateral plate body and the medial plate body to be 4 mm to 4.5 mm.

Further, the design method of an anatomical plate for treating a tibial plateau fracture may further include: obtaining a soft tissue condition around the bone of the patient, and adding bevel angles and fillet angles to the lateral anatomical plate and the medial anatomical plate according to the soft tissue condition in combination with implantation positions of the lateral anatomical plate and the medial anatomical plate; and/or in the simulating intraoperative reduction on the high-precision three-dimensional model to obtain a post-reduction bone model, introducing biomechanical simulation to ensure closure of a fracture line.

According to another aspect of the present disclosure, there is provided an internal fixation device, including a lateral anatomical plate and a medial anatomical plate manufactured in accordance with the design method of an anatomical plate for treating a tibial plateau fracture described above.

With the technical solutions of the present disclosure, since the obtained model matches the anatomic form of the patient, the model is fitter with the state of the patient after recovery. Moreover, the solid body design of the plate body is obtained with the point arrays of the cross section and the sagittal plane; the thicknesses of the proximal ends of the lateral plate body and the medial plate body are then adjusted according to the condition of the soft tissue of the patient; the thicknesses of the main bodies of the lateral plate body and the medial plate body are adjusted according to the body weight of the patient; and the lateral plate body and the medial plate body are cut from the sagittal plane according to the fit condition of the lateral plate body and the medial plate body to obtain the anatomical plate. As such, the design precision of the anatomical plate can be guaranteed, making the anatomical plate match the anatomic form or the fracture form of the patient. Thus, enough stability can be provided, providing stable fixation and being conducive to the healing process of the fracture.

### BRIEF DESCRIPTION OF THE DRAWINGS

The accompany drawings constituting a part of the present disclosure provide further understanding of the present disclosure. The schematic examples of the present disclosure and description thereof are intended to be illustrative of the present disclosure and do not constitute an undue limitation of the present disclosure. In the drawings:

FIG. 1 illustrates a flowchart of a design method of an anatomical plate for treating a tibial plateau fracture provided by an embodiment of the present disclosure;

FIG. 2 illustrates a structural schematic diagram of a post-reduction bone model in a design method of an anatomical plate for treating a tibial plateau fracture provided by an embodiment of the present disclosure;

FIG. 3 illustrates a structural schematic diagram of a post-reduction bone model in a design method of an anatomical plate for treating a tibial plateau fracture provided by an embodiment of the present disclosure;

FIG. 4 illustrates a structural schematic diagram of a post-reduction bone model in a design method of an anatomical plate for treating a tibial plateau fracture provided by an embodiment of the present disclosure;

FIG. 5 illustrates a schematic diagram of point arrays of a cross section and a sagittal plane in a design method of an anatomical plate for treating a tibial plateau fracture provided by an embodiment of the present disclosure;

FIG. 6 illustrates a schematic diagram of a connection curve in a design method of an anatomical plate for treating a tibial plateau fracture provided by an embodiment of the present disclosure;

FIG. 7 illustrates a schematic diagram of a connection curve in a design method of an anatomical plate for treating a tibial plateau fracture provided by an embodiment of the present disclosure;

FIG. 8 illustrates a structural schematic diagram of a lateral plate body in a design method of an anatomical plate for treating a tibial plateau fracture provided by an embodiment of the present disclosure;

FIG. 9 illustrates a structural schematic diagram of a medial plate body in a design method of an anatomical plate for treating a tibial plateau fracture provided by an embodiment of the present disclosure;

FIG. 10 illustrates an exploded diagram of a lateral anatomical plate and a medial anatomical plate in a design method of an anatomical plate for treating a tibial plateau fracture provided by an embodiment of the present disclosure;

FIG. 11 illustrates an exploded diagram of a lateral anatomical plate and a medial anatomical plate in a design method of an anatomical plate for treating a tibial plateau fracture provided by an embodiment of the present disclosure;

FIG. 12 illustrates a structural schematic diagram of a lateral anatomical plate in a design method of an anatomical plate for treating a tibial plateau fracture provided by an embodiment of the present disclosure;

FIG. 13 illustrates a structural schematic diagram of a medial anatomical plate in a design method of an anatomical plate for treating a tibial plateau fracture provided by an embodiment of the present disclosure;

FIG. 14 illustrates an exploded diagram of a lateral anatomical plate and a medial anatomical plate in a design method of an anatomical plate for treating a tibial plateau fracture provided by an embodiment of the present disclosure;

FIG. 15 illustrates an exploded diagram of a lateral anatomical plate and a medial anatomical plate in a design method of an anatomical plate for treating a tibial plateau fracture provided by an embodiment of the present disclosure;

FIG. 16 illustrates an exploded diagram of a lateral anatomical plate and a medial anatomical plate in a design method of an anatomical plate for treating a tibial plateau fracture provided by an embodiment of the present disclosure;

FIG. 17 illustrates an exploded diagram of a lateral anatomical plate and a medial anatomical plate in a design method of an anatomical plate for treating a tibial plateau fracture provided by an embodiment of the present disclosure;

FIG. 18 illustrates a schematic diagram of mounting screws in a lateral anatomical plate and a medial anatomical plate in a design method of an anatomical plate for treating a tibial plateau fracture provided by an embodiment of the present disclosure;

FIG. 19 illustrates a schematic diagram of mounting screws in a lateral anatomical plate and a medial anatomical plate in a design method of an anatomical plate for treating a tibial plateau fracture provided by an embodiment of the present disclosure;

FIG. 20 illustrates a schematic diagram of mounting screws in a lateral anatomical plate and a medial anatomical plate in a design method of an anatomical plate for treating a tibial plateau fracture provided by an embodiment of the present disclosure;

FIG. 21 illustrates a schematic diagram of mounting screws in a lateral anatomical plate and a medial anatomical plate in a design method of an anatomical plate for treating a tibial plateau fracture provided by an embodiment of the present disclosure;

FIG. 22 illustrates a schematic diagram of fitting of a lateral anatomical plate and a medial anatomical plate with a bone in a design method of an anatomical plate for treating a tibial plateau fracture provided by an embodiment of the present disclosure;

FIG. 23 illustrates a schematic diagram of fitting of a lateral anatomical plate and a medial anatomical plate with a bone in a design method of an anatomical plate for treating a tibial plateau fracture provided by an embodiment of the present disclosure;

FIG. 24 illustrates a schematic diagram of fitting of a lateral anatomical plate and a medial anatomical plate with a bone in a design method of an anatomical plate for treating a tibial plateau fracture provided by an embodiment of the present disclosure;

FIG. 25 illustrates a schematic diagram of fitting of a lateral anatomical plate and a medial anatomical plate with a bone in a design method of an anatomical plate for treating a tibial plateau fracture provided by an embodiment of the present disclosure; and

FIG. 26 illustrates a schematic diagram of fitting of a lateral anatomical plate and a medial anatomical plate with a bone in a design method of an anatomical plate for treating a tibial plateau fracture provided by an embodiment of the present disclosure.

Reference numerals in the drawings are as follows:
10, post-reduction bone model;
20, connection curve;
31, lateral plate body; 32, medial plate body;
41, lateral anatomical plate; and 42, medial anatomical plate.

### DETAILED DESCRIPTION OF THE EMBODIMENTS

The technical solutions of the embodiments of the present disclosure are clearly and completely described below with reference to the accompanying drawings. Apparently, the described embodiments are merely a part rather than all of the embodiments of the present disclosure. The following description of at least one exemplary embodiment is merely illustrative, and not intended to limit the present disclosure and application or use thereof in any way. All other embodiments derived from the embodiments of the present disclosure by a person of ordinary skill in the art without creative efforts shall fall within the protection scope of the present disclosure.

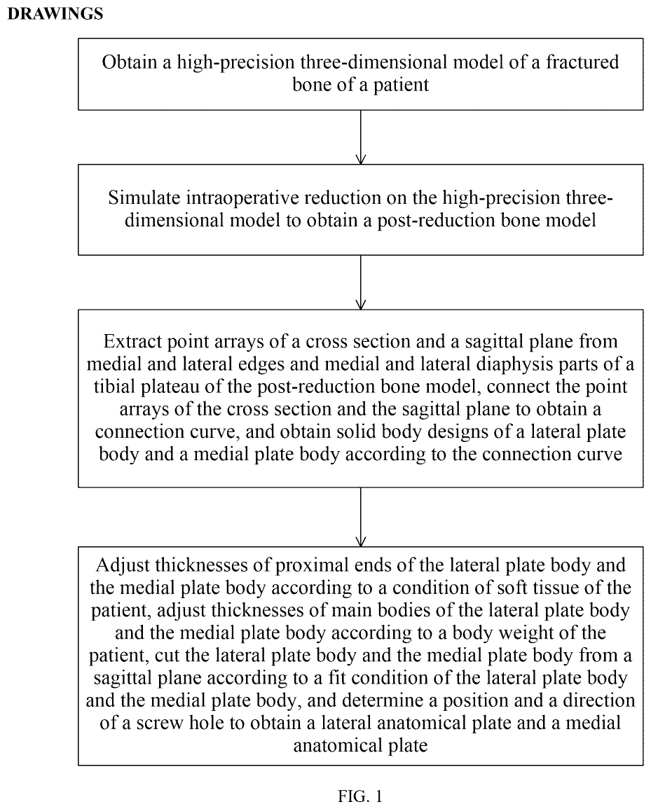

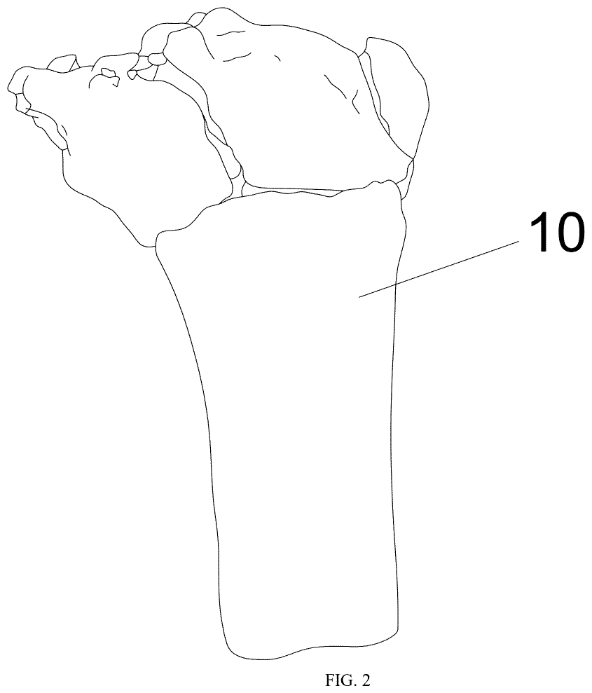

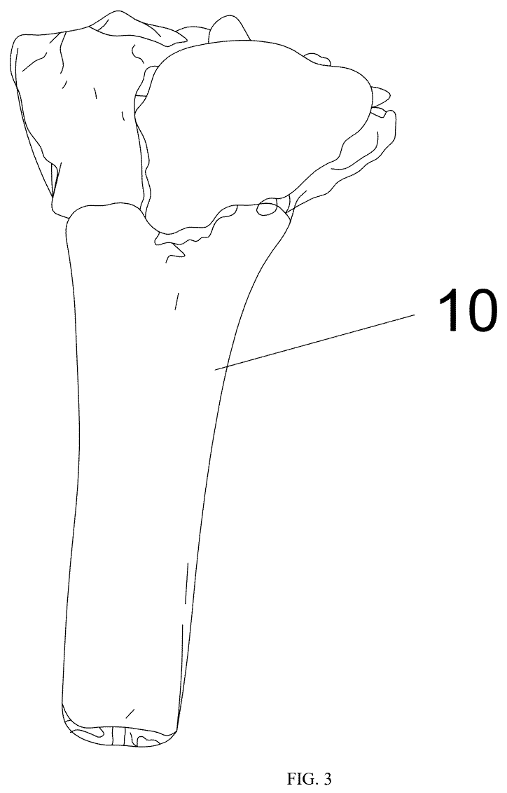

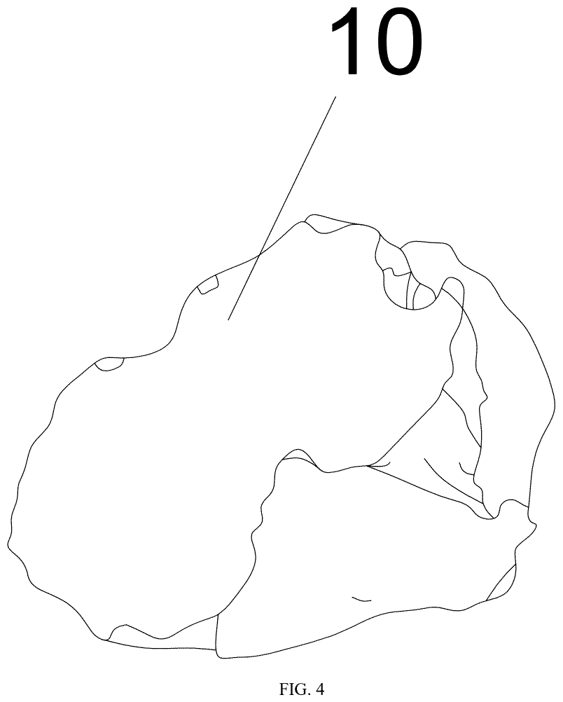

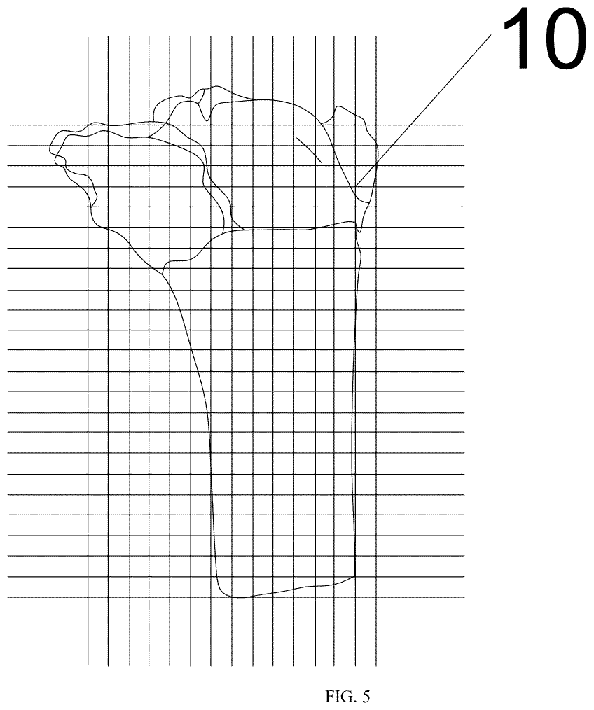

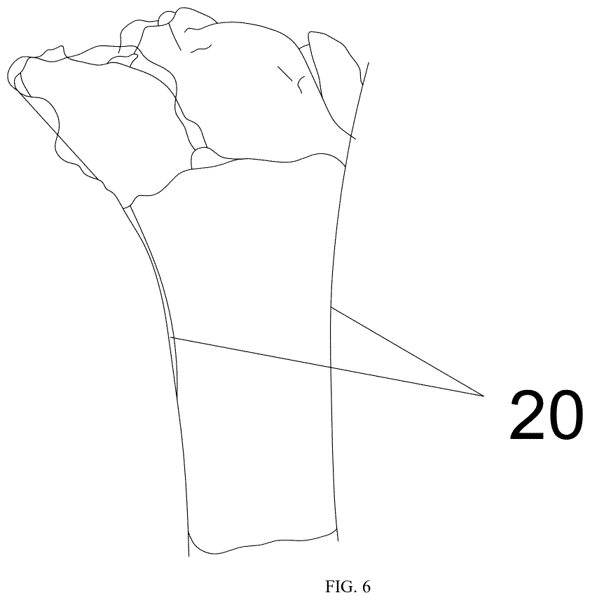

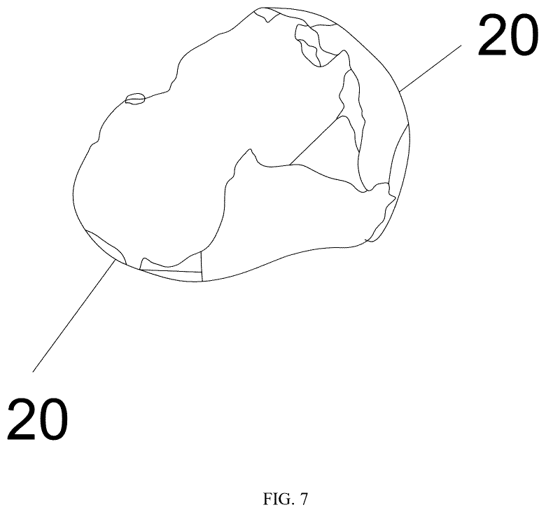

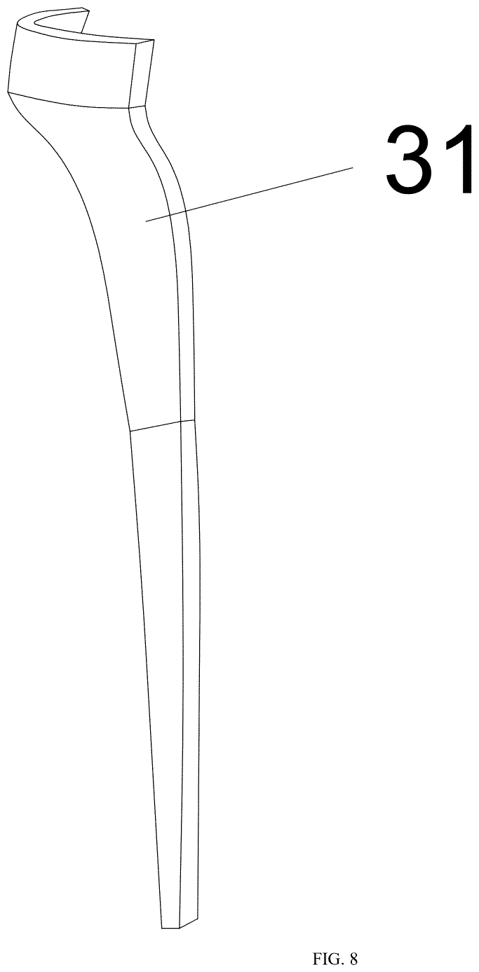

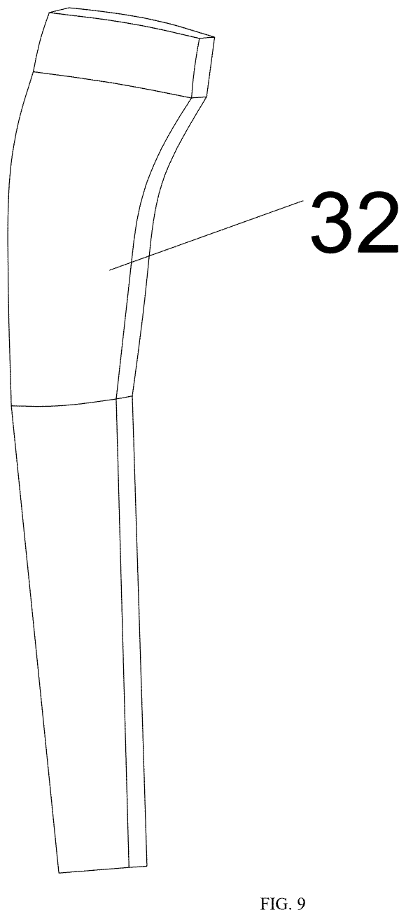

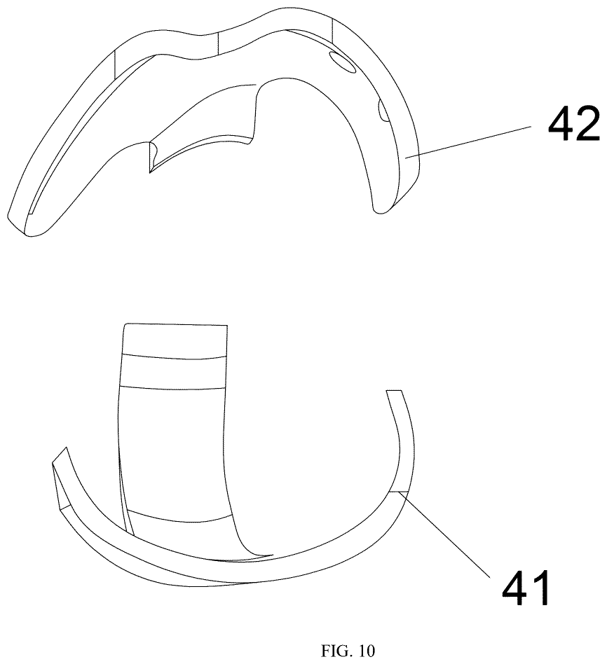

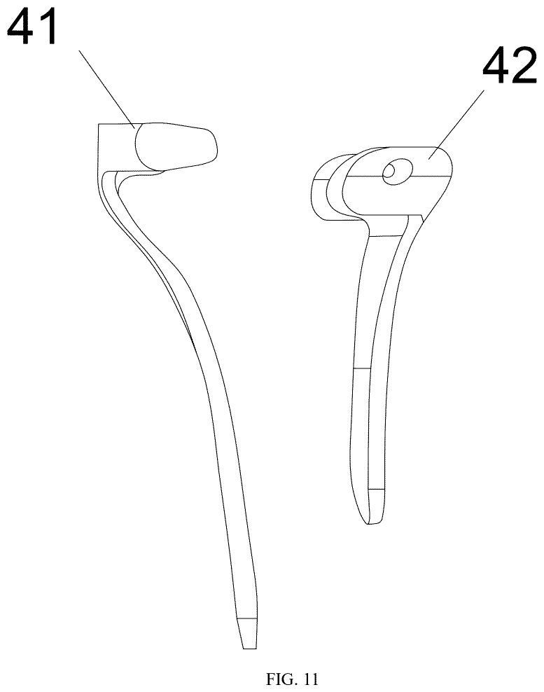

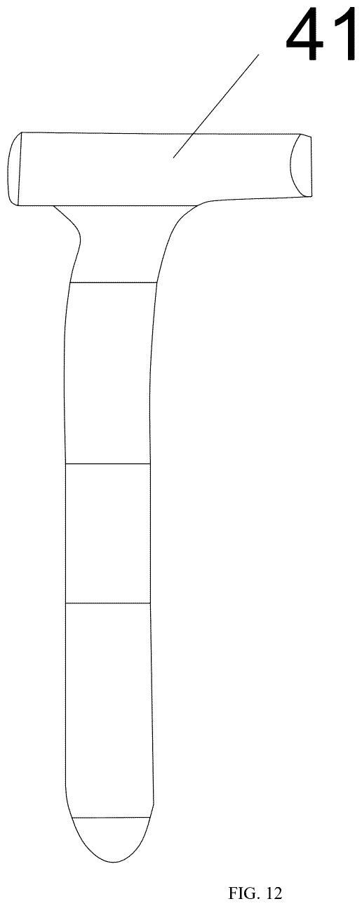

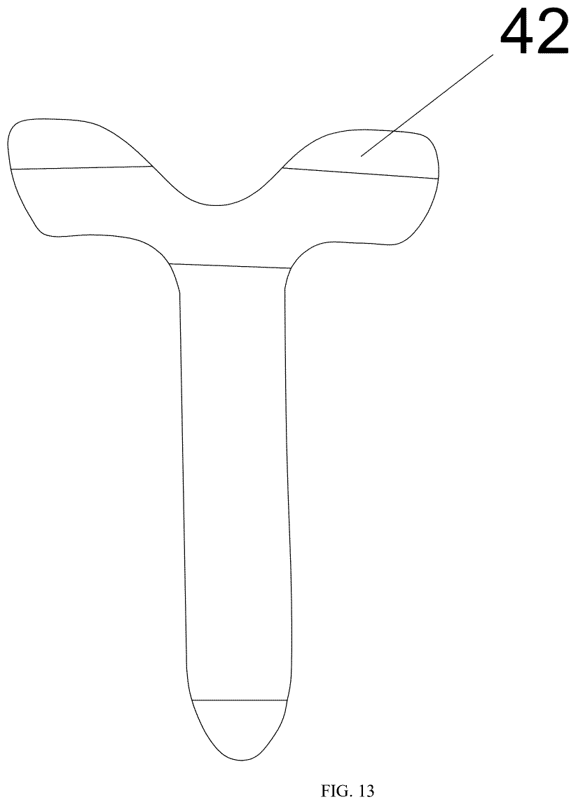

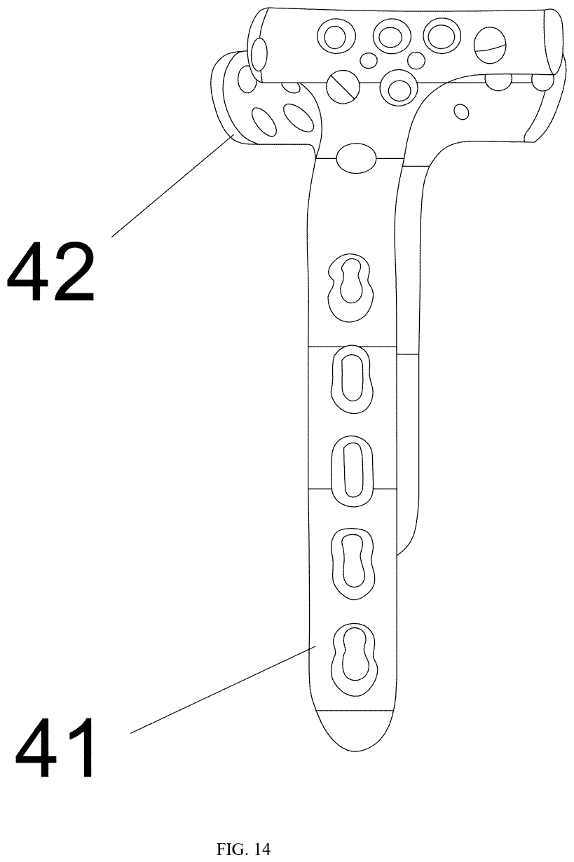

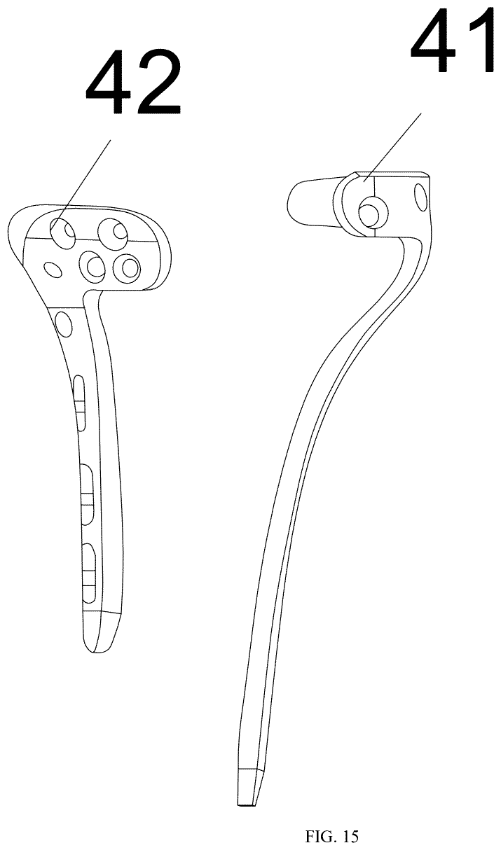

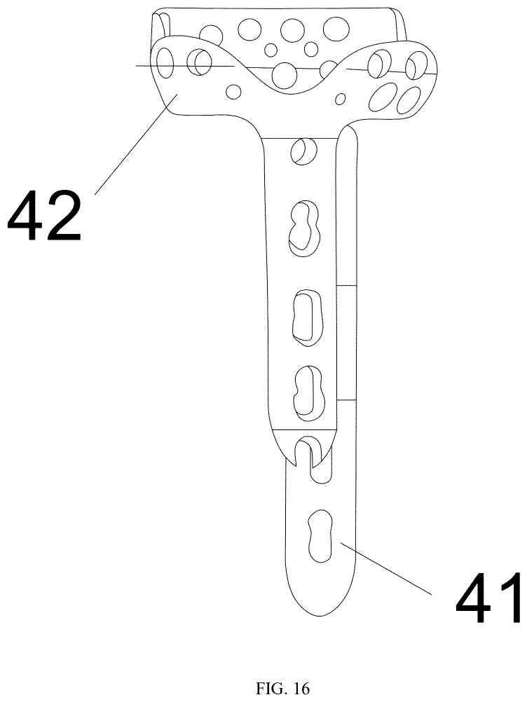

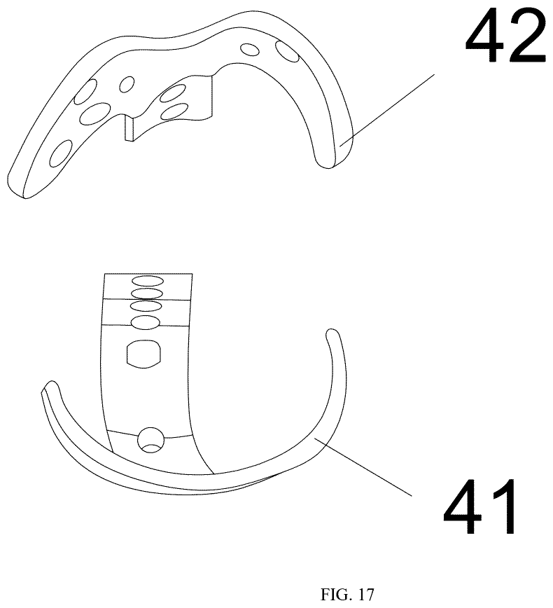

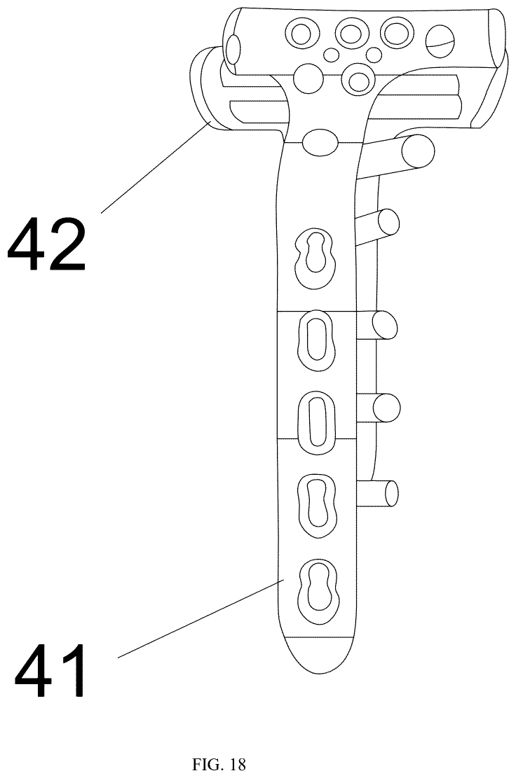

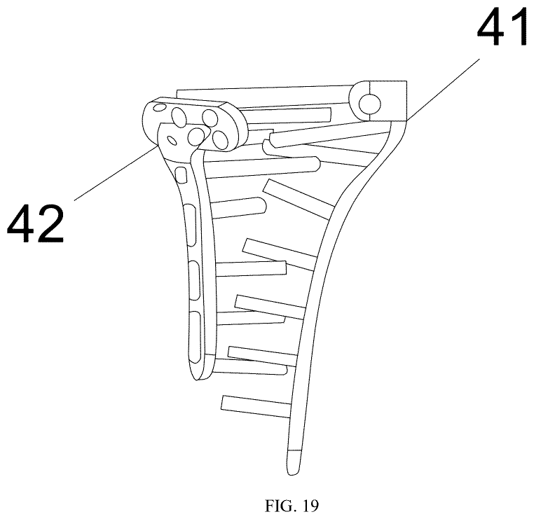

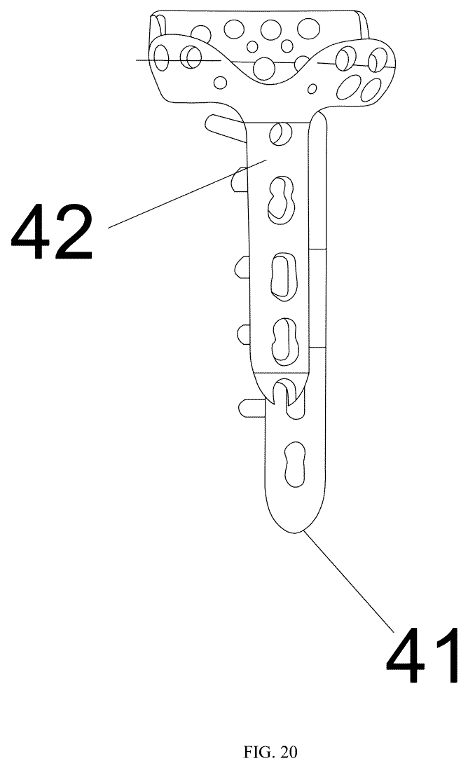

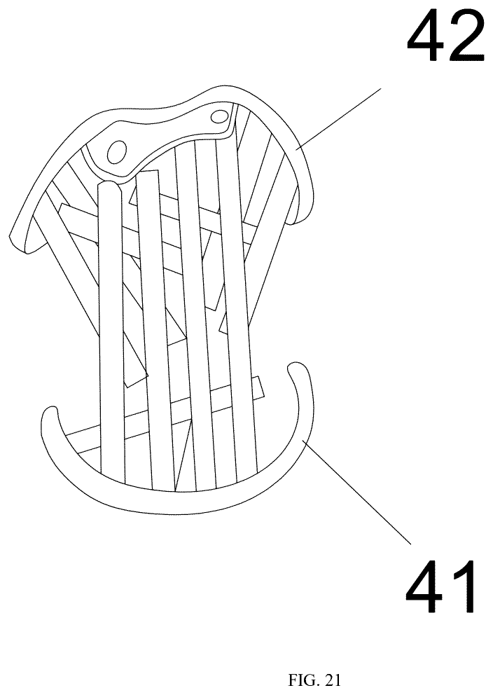

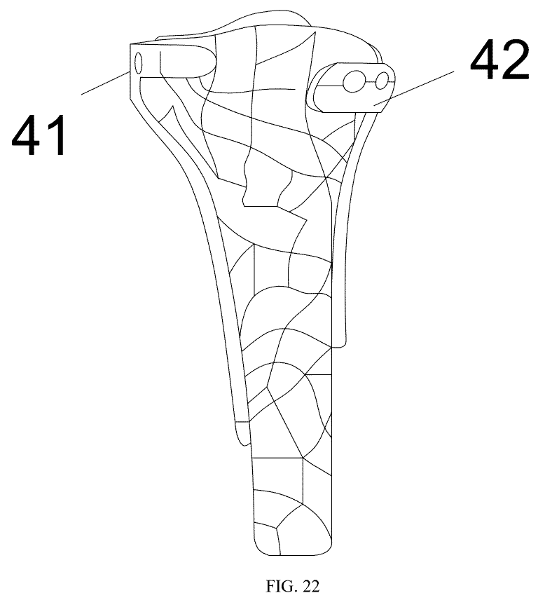

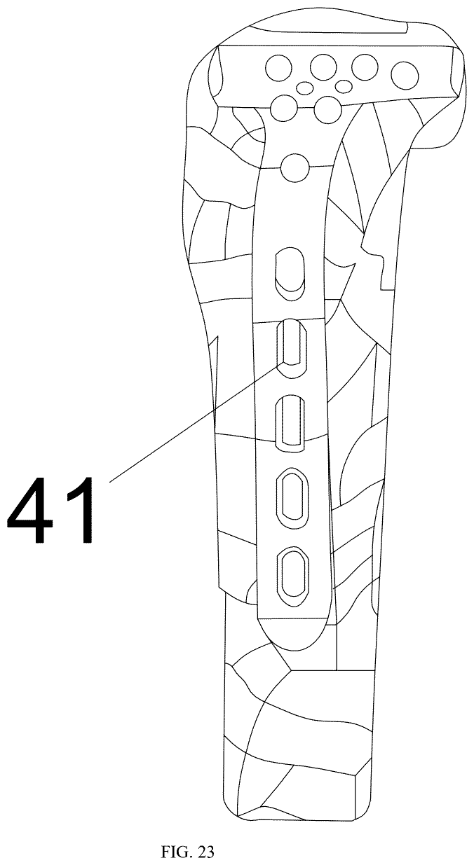

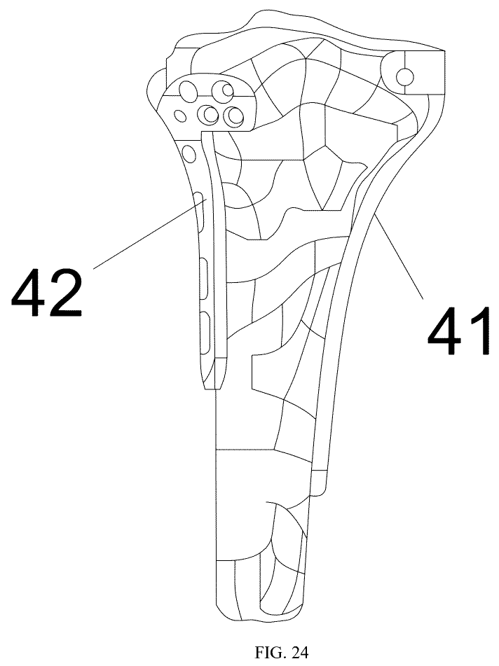

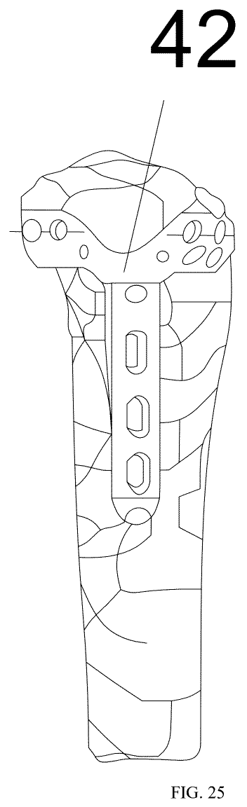

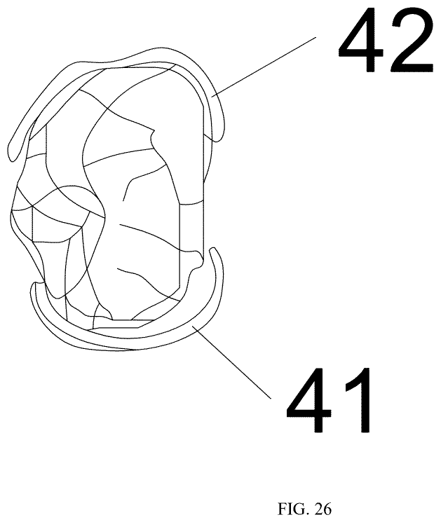

As shown in FIG. 1 to FIG. 26, an embodiment of the present disclosure provides a design method of an anatomical plate for treating a tibial plateau fracture. The design method of an anatomical plate for treating a tibial plateau fracture includes the following steps.

A high-precision three-dimensional model of a fractured bone of a patient is obtained.

Intraoperative reduction is simulated on the high-precision three-dimensional model to obtain a post-reduction bone model 10.

Point arrays of a cross section and a sagittal plane are extracted from medial and lateral edges and medial and lateral diaphysis parts of a tibial plateau of the post-reduction bone model 10; the point arrays of the cross section and the sagittal plane are connected to obtain a connection curve 20; and optimization is made according to an anatomical condition to ensure that a shape and a structure of a solid body part meet more accurate treatment requirements. Solid body designs of a lateral plate body 31 and a medial plate body 32 are obtained according to the connection curve 20.

Thicknesses of proximal ends of the lateral plate body 31 and the medial plate body 32 are adjusted according to a condition of soft tissue of the patient; thicknesses of main bodies of the lateral plate body 31 and the medial plate body 32 are adjusted according to a body weight of the patient; the lateral plate body 31 and the medial plate body 32 are cut from a sagittal plane according to a fit condition of the lateral plate body 31 and the medial plate body 32; and a position and a direction of a screw hole are determined to obtain a lateral anatomical plate 41 and a medial anatomical plate 42.

By the design method of an anatomical plate for treating a tibial plateau fracture provided in this embodiment, since the obtained model matches the anatomic form of the patient, the model is fitter with the state of the patient after recovery. Moreover, the solid body design of the plate body is obtained with the point arrays of the cross section and the sagittal plane; the thicknesses of the proximal ends of the lateral plate body and the medial plate body are then adjusted according to the condition of the soft tissue of the patient; the thicknesses of the main bodies of the lateral plate body and the medial plate body are adjusted according to the body weight of the patient; and the lateral plate body and the medial plate body are cut from the sagittal plane according to the fit condition of the lateral plate body and the medial plate body to obtain the anatomical plate. As such, the design precision of the anatomical plate can be guaranteed, making the anatomical plate match the anatomic form or the fracture form of the patient. Thus, enough stability can be provided, providing stable fixation and being conducive to the healing process of the fracture.

In the related art, a complex tibial plateau fracture repair surgery might increase the risk of postoperative infection, especially when there are many bone fragments and the soft tissue injury is obvious. Moreover, the traditional internal fixation system might require a long-time rehabilitation period, and the function recovery might be limited, thus affecting the living quality of the patient. Since the surgical injury range is expanded, the surrounding tissue might be secondarily injured, causing extra trouble and complications. The design method provided in this embodiment can enable the anatomical plate to match the anatomic form or the fracture form of the patient, simplify the fracture repair surgery, reduce the risk of postoperative infection, shorten the function recovery time, and avoid or reduce secondary injuries.

In this embodiment, a digital imaging and communications in medicine (DICOM) data set of the patient is obtained by imaging techniques such as high-precision computed tomography (CT), and reverse modeling is performed to generate the high-precision three-dimensional bone model of the fractured bone of the patient using a computer. Reduction is performed on the high-precision three-dimensional model of the fractured bone to simulate intraoperative reduction, and biomechanical simulation is introduced to ensure closure of a fracture line while taking the fracture stability and functionality requirements under different postoperative load conditions into account.

A digital imaging and communications in medicine (DICOM) data set of the patient is obtained by imaging techniques such as high-precision computed tomography (CT), and reverse modeling is performed to generate the high-precision three-dimensional bone model of the fractured bone of the patient using a computer. Specific steps are as follows.
a) Obtaining of DICOM data: the DICOM data set of the patient is obtained from a medical imaging device (such as CT scanning, and magnetic resonance imaging (MAI)). The DICOM data includes tomographic image information of the patient.
b) Preprocessing of DICOM data: the DICOM data is preprocessed, including noise removal, image calibration, and slice reconstruction. These preprocessing steps may ensure that high-quality image data is obtained.
c) Three-dimensional reconstruction: the DICOM slices are stacked using a three-dimensional reconstruction algorithm to generate a three-dimensional model of the bone of the patient. Common algorithms include voxel reconstruction, surface reconstruction, etc. These algorithms may convert slice images to consecutive three-dimensional bone surface or voxel representations.
d) Bone segmentation: bone segmentation is performed in the three-dimensional model, and the bone needing to be used is separated from other bones. The bone segmentation may be achieved using a manual segmentation or automatic segmentation algorithm.
e) Three-dimensional model repair and postprocessing: the bone model is repaired and postprocessed, including void filling, surface smoothing, anomaly structure removal, etc. These steps are conducive to generating a complete and accurate bone model.

Reduction is performed on the high-precision three-dimensional model of the fractured bone to simulate intraoperative reduction, and biomechanical simulation is introduced to ensure the closure of the fracture line while taking the fracture stability and functionality requirements under different postoperative load conditions into account. The three-dimensional model of the fractured bone is reduced using computer aided design (CAD) software or a specialized medical image processing tool. This includes recovering the fracture line to the normal anatomical position and ensuring the closure of the fracture line. For a patient with a tibial plateau defect, if bone blocks for reverse modeling are incomplete, the integrity of the outer contour of the tibial plateau and that of the tibial shaft need to be guaranteed.

The point arrays of the cross section and the coronal plane are extracted from the high-precision three-dimensional model of the medial and lateral edges and the medial and lateral diaphysis parts of the tibial plateau, and a coronal plane and cross section reference plane is established. Intersection points of the coronal plane and cross section reference plane with the bone model are the point arrays needing to be picked up. The point arrays of the cross section and the coronal plane are connected such that curves can be obtained by fitting. The curves of different planes are connected to form a sheet body, and optimization is made according to the anatomical condition to ensure that the shape and the structure after fitting meet the more accurate treatment requirements. Specific steps are as follows.

Generation of point arrays: a series of points in regions needing to be connected are selected on the high-precision three-dimensional model of the cross section and the coronal plane. These points should cover the whole connected region, and the anatomical structure and the fracture condition of the patient are taken into account. The density and positions of the points should be selected as needed.

Generation of connection curve: the selected points are used to generate the connection curve by interpolation or other mathematical methods. These curves will connect the point arrays of different planes to form smooth transition. This step usually involves the CAD software or three-dimensional modeling tool.

Formation of connection curve into sheet body: the connection curve is scanned to form a sheet body. That is, an enclosed solid body is created around the connection curve. This may be achieved by extending the cross section of the curve along the curve path. This process will generate the basic shape of the sheet body.

Anatomical optimization: once the sheet body is generated, anatomical optimization may be made thereto. This includes fine adjusting the shape and the structure of the sheet body to ensure it meets the more accurate treatment requirements.

In this embodiment, the step of cutting the lateral plate body 31 and the medial plate body 32 from a sagittal plane according to a fit condition of the lateral plate body 31 and the medial plate body 32 includes the following steps. The lateral plate body 31 is cut into a T shape in the sagittal plane, and both wings of the lateral plate body 31 are designed to form a C-shaped cross section. The medial plate body 32 is cut into a γ shape in the sagittal plane, and both wings of the medial plate body 32 are designed to extend inwards and a middle part of the medial plate body 32 to be a recess for escaping medial condyle process. The step of cutting the lateral plate body 31 and the medial plate body 32 from a sagittal plane according to a fit condition of the lateral plate body 31 and the medial plate body 32 further includes the following steps. An upper edge of the lateral plate body 31 is designed to be flush with an upper edge of the tibial plateau; and an upper edge of the medial plate body 32 is designed to be lower than the upper edge of the lateral plate body 31.

In this embodiment, the step of determining a position and a direction of a screw hole includes the following steps. Two rows of first screw holes are designed at the proximal end of the lateral plate body 31; an obliquely upward second screw hole is designed at a first screw hole in a neck of the lateral plate body 31; a third screw hole is designed in the main body of the lateral plate body 31. Specifically, the step of determining a position and a direction of a screw hole further includes the following steps. Two rows of fourth screw holes extending obliquely backwards from front are designed at a front side of the medial plate body 32; a fifth screw hole is designed at a rear side of the medial plate body 32; an obliquely upward sixth screw hole is designed at a first screw hole in a neck of the medial plate body 32; a seventh screw hole is designed in the main body of the medial plate body 32.

The step of determining a position and a direction of a screw hole further includes the following steps. An included angle between an extension direction of the fourth screw hole and a horizontal axis of the tibial plateau is designed to be less than or equal to 30°; and/or the two rows of first screw holes and the two rows of fourth screw holes are designed to be staggered up and down.

In this embodiment, the thickened body is cut from the sagittal plane according to the part needing to be fit and fixed so as to fix different bone fragments.

Observed from the sagittal plane, the lateral plate (the lateral plate body 31) is T-shaped and thus can fix and support different parts of the tibial plateau through front-and-back extension of the proximal end according to the fracture type. The lateral plate has a C-shaped cross section and can surround the tibial plateau. Its upper edge is substantially flush with the upper edge of the tibial plateau, providing support. Meanwhile, the main body is connected and fixed to enhance the overall stability. The lateral plate may extend towards the front side or the rear side, and the extension length may be customized according to the condition of the plateau of the patient, which is about ¼ of the transverse diameter of the tibial plateau. The width of the part extending backwards is designed according to the upper edge of the fibula and the tibial plateau. The design should be kept away from the position of the fibula. The front edge may be slightly wide, about 10 mm, and a row of locking screws may be placed at the front edge. The front side and the rear side of the upper edge should be low-profile as much as possible, and the thickness is about 1.5-2 mm. The middle neck is a transitional region, which is transitional in width and thickness. The thickness and the width of the main body are designed with reference to a standard plate.

The medial plate (the medial plate body 32) is γ-shaped in the sagittal plane and has moderately inward wing surfaces on both sides. The tibial medial plateau is jointly lifted from the front side and the rear side, preventing reduction fixation from moving down. The medial plate has a certain inwardly recessed radian in the sagittal plane. The inwardly recessed radian and a distance to an upper edge are determined according to the geometric outline of the tibial medial plateau. The medial plate can lift the medial plateau. The middle region is recessed inwardly to escape the medial condyle process, reducing profile damage to the soft tissue. The upper edge is slightly lower than the lateral plate and may be staggered from screw arranging holes of the lateral plate, thereby increasing the screw arrangement space.

With the shape design of medial and lateral plates, the bone block of the tibial plateau can be fixed roundly while reducing the soft tissue injury. The shape of the plate is highly fit with the anatomical structure of the human body, which is conducive to improving the surgical efficiency and the recovery effect. With the optimization design of the shapes, the structures, and the orientations of two plates, customized support and fixation may be provided for different parts of the tibial plateau. Such a personalized design is helpful for treating a complex fracture and increasing the success rate of surgery.

Specifically, according to the conditions of the bone fragments and the mechanical performance results, the optimal positions of the screw holes are determined on the high-precision model and the direction of the screw holes is locked.

A two-row “bamboo raft” screw arrangement is designed at the proximal end of the lateral plate to ensure effective support for the lateral plateau, and the force acting on all screws can be averaged to prevent stress concentration. On the front side, it is designed that a screw is driven backwards to realize fixation in the front-and-back direction. The first screw for the neck is driven obliquely upwards, and the design is made according to this position and the anatomic form of the tibial plateau of the patient to realize oblique driving at a maximum angle for effectively supporting the tibial plateau. The screws for the main body are arranged with reference to a conventional manner, and a composite screw hole is adopted. That is, a lag screw may be used, or a locking screw may also be used. The screw holes are provided at intervals of 5-10 mm, and the length is generally about 3-4 screw holes away from the farthest fracture line.

Two rows of screws are provided at the front edge of the medial plate, which are driven obliquely backwards from the front, and the direction should be at an angle of less than 30° with the horizontal axis of the tibial plateau, facilitating intraoperative screw implantation. The two rows of screws should be staggered up and down with the two rows of screws of the lateral plate so as to realize multi-dimensional planar fixation. The direction of the screws on the rear side is at the angle of less than 30° with the horizontal axis of the tibial plateau, facilitating intraoperative screw implantation. The first screw for the neck is driven obliquely upwards, and the design is made according to this position and the anatomic form of the tibial plateau of the patient to realize oblique driving at a maximum angle for effectively supporting the tibial plateau. The screws for the main body are arranged with reference to a conventional manner, and a composite screw hole is adopted. That is, a lag screw may be used, or a locking screw may also be used. The screw holes are provided at intervals of 5-10 mm, and the length is generally about 3-4 screw holes away from the farthest fracture line.

The arrangement of the screw holes needs to follow the following principle: the positions of the bone fragments are identified according to CT, and points are selected on two sides of the fracture line for designing screw holes. The design of the screw holes needs to cover the bone fragments. However, the screw holes should not be too dense, thus avoiding influence on the intrabony blood supply. The screw holes of the proximal end and the distal end are 5-10 mm away from end faces, keeping away from the joint surface. A “bamboo raft” type screw hole layout is designed for the platform near the joint. The screw holes are in a mesh layout. The holes are arranged at intervals of 10-15 mm. such a layout may averagely dispersing the load on the screws. The screw holes in the main bodies are designed to be staggered up and down, thereby enhancing the torsional strength and the bending strength of the main bodies. The fixation of the front and rear cortical layers of the diaphysis shall be taken into account in screw setting. The screw holes in the main bodies of the medial and lateral plates are designed to be staggered up and down. The fixation of the front and rear cortical layers of the diaphysis can be realized.

According to the force conditions of different parts, the postoperative suture requirement, and the biomechanical simulation result, the sheet body thicknesses and shapes of different parts are adjusted to obtain the optimal solid body design. The thicknesses of the proximal end and the distal end of the plate are adjusted according to different requirements of the proximal end and the distal end of the tibia.

In consideration of the individual difference of the patient, the thickness of the proximal region needs to be designed with respect to the condition of the soft tissue.

In this embodiment, the step of adjusting the thicknesses of the proximal ends of the lateral plate body 31 and the medial plate body 32 according to the condition of soft tissue of the patient includes the following steps.

If a thickness of the soft tissue of the patient is less than 20 mm, the thicknesses of the proximal ends of the lateral lateral plate body 31 and the medial plate body 32 are set to be 2 mm to 2.5 mm. When the thickness of the soft tissue of the patient is less than 20 mm, it is easy to cause exposure and infection. A thinner plate needs to be used, for example, about 2 mm.

If the thickness of the soft tissue of the patient is 20 mm to 30 mm, a standard thickness is adopted, and the thicknesses of the proximal ends of the lateral plate body 31 and the medial plate body 32 are set to be 2.5 mm to 3 mm, such as 2.5 mm, 2.75 mm, and 3 mm.

If the thickness of the soft tissue of the patient is greater than 30 mm, the thicknesses of the proximal ends of the lateral plate body 31 and the medial plate body 32 are set to be 3 mm to 3.5 mm, such as 3 mm, 3.25 mm, and 3.5 mm, to enhance the fixation strength.

Main body region: the main body is a stress concentration region and needs to be optimized in design to prevent breakage. In consideration of the influence of the body weight of a patient, the stress of an overweight patient is greater and a thicker plate is required.

In this embodiment, the step of adjusting the thicknesses of the main bodies of the lateral plate body 31 and the medial plate body 32 according to the body weight of the patient includes the following steps.

If the body weight of the patient is less than 60 kg, the thicknesses of the main bodies of the lateral plate body 31 and the medial plate body 32 are set to be 3 mm to 3.5 mm, such as 3 mm, 3.25 mm, and 3.5 mm, to obtain the basic bending strength.

If the body weight of the patient is greater than or equal to 60 kg and less than or equal to 90 kg, the thicknesses of the main bodies of the lateral plate body 31 and the medial plate body 32 are set to be 3.5 mm to 4 mm, such as 3.5 mm, 3.75 mm, and 4 mm, to increase the bending strength.

If the body weight of the patient is greater than 90 kg, the thicknesses of the main bodies of the lateral plate body 31 and the medial plate body 32 are set to be 4 mm to 4.5 mm, such as 4 mm, 4.25 mm, and 4.5 mm, to significantly improve the bending strength and ensure the fixation effect.

The design method of an anatomical plate for treating a tibial plateau fracture further includes the following steps. A soft tissue condition around the bone of the patient is obtained, and bevel angles and fillet angles are added to the lateral anatomical plate 41 and the medial anatomical plate 42 according to the soft tissue condition in combination with implantation positions of the lateral anatomical plate 41 and the medial anatomical plate 42. In consideration of the surrounding soft tissue and the position of an implant, high-precision design of the plate is carried out to ensure the optimization operation in the surgical process.

The bevel angle design is added to the edge of the plate, and small bevel angles are adopted for transition at the joints of the front and rear ends with the flanges of the plate. This may avoid compression of the soft tissue by the plate angles, thereby reducing the wound tension and facilitating wound closure. The circular arc transition is added to the flange parts of the plate, and circular-arc-shaped smooth transition is provided for the contact surface of the plate and the bone. This may reduce the friction of the soft tissue caused by the edges of the plate, preventing tissue tearing after closure. The surface smoothness of the plate is optimized by using the precise molding process, and the surface roughness Ra is controlled to be less than 0.5 μm. The smooth surface may reduce the inflammatory response and the infection risk of the wound.

In this embodiment, in the step of simulating intraoperative reduction on the high-precision three-dimensional model to obtain the post-reduction bone model 10, biomechanical simulation is introduced to ensure the closure of the fracture line.

Another embodiment of the present disclosure provides an internal fixation device, including a lateral anatomical plate 41 and a medial anatomical plate 42 manufactured in accordance with the design method of an anatomical plate for treating a tibial plateau fracture described above. Therefore, the internal fixation device can also guarantee the design precision of the anatomical plate such that the anatomical plate matches the anatomic form or the fracture form of a patient, and thus can provide enough stability, leading to stable fixation and being conducive to the healing process of the fracture.

Specifically, the shape and the size of the internal fixation device may be customized according to the fracture condition and the anatomical structure of a specific patient. The dislocation condition of a plurality of bone fragments is taken into full consideration in the design to provide three-dimensional positioning stability. The internal fixation strength may allow for fixation in a plurality of planes to ensure multi-dimensional stable support. For the length and the shape, the requirement of the operative route and the postoperative suture condition are taken into full consideration. The design takes the biomechanical requirement of fracture healing into full consideration to facilitate faster healing and the rehabilitation of the patient.

It should be noted that the terminology used herein is for the purpose of describing particular embodiments only and is not intended to limit exemplary embodiments according to the present disclosure. As used herein, unless otherwise specified herein, the singular forms are also intended to include the plural forms. In addition, it should also be understood that when the terms “comprise” and/or “include” are used in this specification, they specify the presence of features, steps, operations, devices, components, and/or combinations thereof.

Unless otherwise specified, the relative arrangement, numerical expressions and numerical values of components and steps set forth in these examples do not limit the scope of the present disclosure. Meanwhile, it should be understood that for ease of description, each portion in the drawings is not necessarily drawn to the actual scale. The technologies, methods, and devices known to those of ordinary skill in the art may not be discussed in detail, but where appropriate, the technologies, methods, and devices should be regarded as part of the specification. In all examples shown and discussed herein, any specific value should be interpreted as merely exemplary, rather than restrictive. Therefore, other examples of the exemplary embodiments may have different values. It should be noted that similar reference signs and letters represent similar items in the accompanying drawings below. Therefore, once an item is defined in one drawing, it does not need to be further discussed in subsequent drawings.

It should be understood that, in the description of the present disclosure, terms such as “front”, “rear”, “upper”, “lower”, “left”, “right”, “transverse”, “longitudinal”, “vertical”, “horizontal”, “top” and “bottom” indicate orientation or position relationships based on the accompanying drawings. Unless otherwise specified, these terms are merely intended to facilitate or simplify the description of the present disclosure, rather than to indicate or imply that the mentioned device or components must have a specific orientation and must be constructed and operated in a specific orientation. Therefore, they should not be construed as a limitation to the protection scope of the present disclosure. The orientation terms “inner” and “outer” refer to the inner and outer parts relative to the contour of the mentioned component.

For ease of description, spatially relative terms, such as “above”, “on the upper side of”, “on the upper surface of” and “on”, can be used to describe the spatial positional relationship between components or features shown in the figure. It should be understood that the spatially relative terms are intended to encompass different orientations of the components in use or operation in addition to those shown in the figure. For example, if a component in the figure is inverted, it is described as a component “above other component or structure” or “on other component or structure”. Therefore, the component will be positioned as “below other component or structure” or “under other component or structure”. Therefore, the exemplary term “above” may include both orientations “above” and “below”. The component may also be positioned in other different ways (rotated by 90 degrees or in other orientations), but the relative description of the space should be explained accordingly.

In addition, it needs to be noted that the use of such words as “first” and “second” to define components is merely intended to distinguish the corresponding components. Unless otherwise stated, such words have no special meaning and thus cannot be construed as limiting the protection scope of the present disclosure.

The foregoing are merely descriptions of the preferred embodiments of the present disclosure and not intended to limit the present disclosure, and various changes and modifications of the present disclosure may be made by those skilled in the art. Any modifications, equivalent substitutions, improvements and the like made within the spirit and principle of the present disclosure should be included within the protection scope of the present disclosure.

1. A design method of an anatomical plate for treating a tibial plateau fracture, comprising:
obtaining a high-precision three-dimensional model of a fractured bone of a patient;
simulating intraoperative reduction on the high-precision three-dimensional model to obtain a post-reduction bone model (10);
extracting point arrays of a cross section and a sagittal plane from medial and lateral edges and medial and lateral diaphysis parts of a tibial plateau of the post-reduction bone model (10), connecting the point arrays of the cross section and the sagittal plane to obtain a connection curve (20), and obtaining solid body designs of a lateral plate body (31) and a medial plate body (32) according to the connection curve (20); and
adjusting thicknesses of proximal ends of the lateral plate body (31) and the medial plate body (32) according to a condition of soft tissue of the patient, adjusting thicknesses of main bodies of the lateral plate body (31) and the medial plate body (32) according to a body weight of the patient, cutting the lateral plate body (31) and the medial plate body (32) from a sagittal plane according to a fit condition of the lateral plate body (31) and the medial plate body (32), and determining a position and a direction of a screw hole to obtain a lateral anatomical plate (41) and a medial anatomical plate (42).

2. The design method of an anatomical plate for treating a tibial plateau fracture according to claim 1, wherein the cutting the lateral plate body (31) and the medial plate body (32) from a sagittal plane according to a fit condition of the lateral plate body (31) and the medial plate body (32) comprises:
cutting the lateral plate body (31) into a T shape in the sagittal plane, and designing both wings of the lateral plate body (31) forming a C-shaped cross section; and
cutting the medial plate body (32) into a γ shape in the sagittal plane, and designing both wings of the medial plate body (32) to extend inwards and a middle part of the medial plate body (32) to be a recess for escaping medial condyle process.

3. The design method of an anatomical plate for treating a tibial plateau fracture according to claim 2, wherein the cutting the lateral plate body (31) and the medial plate body (32) from a sagittal plane according to a fit condition of the lateral plate body (31) and the medial plate body (32) further comprises:
designing an upper edge of the lateral plate body (31) to be flush with an upper edge of the tibial plateau; and
designing an upper edge of the medial plate body (32) to be lower than the upper edge of the lateral plate body (31).

4. The design method of an anatomical plate for treating a tibial plateau fracture according to claim 2, wherein the determining a position and a direction of a screw hole comprises:
designing two rows of first screw holes at the proximal end of the lateral plate body (31), designing an obliquely upward second screw hole at a first screw hole in a neck of the lateral plate body (31), and designing a third screw hole in the main body of the lateral plate body (31).

5. The design method of an anatomical plate for treating a tibial plateau fracture according to claim 4, wherein the determining a position and a direction of a screw hole further comprises:
designing two rows of fourth screw holes extending obliquely backwards from front at a front side of the medial plate body (32), designing a fifth screw hole at a rear side of the medial plate body (32), designing an obliquely upward sixth screw hole at a first screw hole in a neck of the medial plate body (32), and designing a seventh screw hole in the main body of the medial plate body (32).

6. The design method of an anatomical plate for treating a tibial plateau fracture according to claim 5, wherein the determining a position and a direction of a screw hole further comprises:
designing an included angle between an extension direction of the fourth screw hole and a horizontal axis of the tibial plateau to be less than or equal to 30°; and/or
designing the two rows of first screw holes and the two rows of fourth screw holes to be staggered up and down.

7. The design method of an anatomical plate for treating a tibial plateau fracture according to claim 1, wherein the adjusting thicknesses of proximal ends of the lateral plate body (31) and the medial plate body (32) according to a condition of soft tissue of the patient comprises:
if a thickness of the soft tissue of the patient is less than 20 mm, setting the thicknesses of the proximal ends of the lateral plate body (31) and the medial plate body (32) to be 2 mm to 2.5 mm;
if the thickness of the soft tissue of the patient is 20 mm to 30 mm, setting the thicknesses of the proximal ends of the lateral plate body (31) and the medial plate body (32) to be 2.5 mm to 3 mm; and
if the thickness of the soft tissue of the patient is greater than 30 mm, setting the thicknesses of the proximal ends of the lateral plate body (31) and the medial plate body (32) to be 3 mm to 3.5 mm.

8. The design method of an anatomical plate for treating a tibial plateau fracture according to claim 1, wherein the adjusting thicknesses of main bodies of the lateral plate body (31) and the medial plate body (32) according to a body weight of the patient comprises:
if the body weight of the patient is less than 60 kg, setting the thicknesses of the main bodies of the lateral plate body (31) and the medial plate body (32) to be 3 mm to 3.5 mm;
if the body weight of the patient is greater than or equal to 60 kg and less than or equal to 90 kg, setting the thicknesses of the main bodies of the lateral plate body (31) and the medial plate body (32) to be 3.5 mm to 4 mm; and
if the body weight of the patient is greater than 90 kg, setting the thicknesses of the main bodies of the lateral plate body (31) and the medial plate body (32) to be 4 mm to 4.5 mm.

9. The design method of an anatomical plate for treating a tibial plateau fracture according to claim 1, further comprising:
obtaining a soft tissue condition around the bone of the patient, and adding bevel angles and fillet angles to the lateral anatomical plate (41) and the medial anatomical plate (42) according to the soft tissue condition in combination with implantation positions of the lateral anatomical plate (41) and the medial anatomical plate (42); and/or
in the simulating intraoperative reduction on the high-precision three-dimensional model to obtain a post-reduction bone model (10), introducing biomechanical simulation to ensure closure of a fracture line.

10. An internal fixation device, comprising a lateral anatomical plate (41) and a medial anatomical plate (42) manufactured in accordance with the design method of an anatomical plate for treating a tibial plateau fracture according to claim 1.

11. The internal fixation device according to claim 10, wherein the cutting the lateral plate body (31) and the medial plate body (32) from a sagittal plane according to a fit condition of the lateral plate body (31) and the medial plate body (32) comprises:
cutting the lateral plate body (31) into a T shape in the sagittal plane, and designing both wings of the lateral plate body (31) forming a C-shaped cross section; and
cutting the medial plate body (32) into a γ shape in the sagittal plane, and designing both wings of the medial plate body (32) to extend inwards and a middle part of the medial plate body (32) to be a recess for escaping medial condyle process.

12. The internal fixation device according to claim 11, wherein the cutting the lateral plate body (31) and the medial plate body (32) from a sagittal plane according to a fit condition of the lateral plate body (31) and the medial plate body (32) further comprises:
designing an upper edge of the lateral plate body (31) to be flush with an upper edge of the tibial plateau; and
designing an upper edge of the medial plate body (32) to be lower than the upper edge of the lateral plate body (31).

13. The internal fixation device according to claim 11, wherein the determining a position and a direction of a screw hole comprises:
designing two rows of first screw holes at the proximal end of the lateral plate body (31), designing an obliquely upward second screw hole at a first screw hole in a neck of the lateral plate body (31), and designing a third screw hole in the main body of the lateral plate body (31).

14. The internal fixation device according to claim 13, wherein the determining a position and a direction of a screw hole further comprises:
designing two rows of fourth screw holes extending obliquely backwards from front at a front side of the medial plate body (32), designing a fifth screw hole at a rear side of the medial plate body (32), designing an obliquely upward sixth screw hole at a first screw hole in a neck of the medial plate body (32), and designing a seventh screw hole in the main body of the medial plate body (32).

15. The internal fixation device according to claim 14, wherein the determining a position and a direction of a screw hole further comprises:
designing an included angle between an extension direction of the fourth screw hole and a horizontal axis of the tibial plateau to be less than or equal to 30°; and/or
designing the two rows of first screw holes and the two rows of fourth screw holes to be staggered up and down.

16. The internal fixation device according to claim 10, wherein the adjusting thicknesses of proximal ends of the lateral plate body (31) and the medial plate body (32) according to a condition of soft tissue of the patient comprises:
if a thickness of the soft tissue of the patient is less than 20 mm, setting the thicknesses of the proximal ends of the lateral plate body (31) and the medial plate body (32) to be 2 mm to 2.5 mm;
if the thickness of the soft tissue of the patient is 20 mm to 30 mm, setting the thicknesses of the proximal ends of the lateral plate body (31) and the medial plate body (32) to be 2.5 mm to 3 mm; and
if the thickness of the soft tissue of the patient is greater than 30 mm, setting the thicknesses of the proximal ends of the lateral plate body (31) and the medial plate body (32) to be 3 mm to 3.5 mm.

17. The internal fixation device according to claim 10, wherein the adjusting thicknesses of main bodies of the lateral plate body (31) and the medial plate body (32) according to a body weight of the patient comprises:
if the body weight of the patient is less than 60 kg, setting the thicknesses of the main bodies of the lateral plate body (31) and the medial plate body (32) to be 3 mm to 3.5 mm;
if the body weight of the patient is greater than or equal to 60 kg and less than or equal to 90 kg, setting the thicknesses of the main bodies of the lateral plate body (31) and the medial plate body (32) to be 3.5 mm to 4 mm; and
if the body weight of the patient is greater than 90 kg, setting the thicknesses of the main bodies of the lateral plate body (31) and the medial plate body (32) to be 4 mm to 4.5 mm.

18. The internal fixation device according to claim 10, further comprising:
obtaining a soft tissue condition around the bone of the patient, and adding bevel angles and fillet angles to the lateral anatomical plate (41) and the medial anatomical plate (42) according to the soft tissue condition in combination with implantation positions of the lateral anatomical plate (41) and the medial anatomical plate (42); and/or
in the simulating intraoperative reduction on the high-precision three-dimensional model to obtain a post-reduction bone model (10), introducing biomechanical simulation to ensure closure of a fracture line.
